(19) United States
(12) Patent Application Publication
(10) Pub. No.: US 2005/0197596 A1
(43) Pub. Date: Sep. 8, 2005

(54) Umbilical cord sampling system and method

(76) Inventors: Mitchell J. Bellucci, Milford, MA (US); Richard A. Smith, Attleboro, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/938,745

(22) Filed: Sep. 10, 2004

Related U.S. Application Data
(63) Continuation-in-part of application No. 10/660,102, filed on Sep. 10, 2003.

(51) Int. Cl. A61B 5/00

(52) U.S. Cl. 600/573

(57) ABSTRACT

The system and method of the present invention includes an umbilical cord sampling device comprising a needle assembly, a base and at least one sampling needle operatively linked to at least one sampling reservoir. A removable cassette can be used to transfer a fluid to an analyzer to provide for the measurement of fluid characteristics.

### CROSS REFERENCES TO RELATED APPLICATIONS

The present application is a continuation-in-part of co-pending U.S. patent application Ser. No. 10/660,102, filed Sep. 10, 2003. The entire contents of the above application is incorporated herein by reference.

### BACKGROUND OF THE INVENTION

Rapid analysis of physiologically relevant parameters of neonatal blood provides useful information for decisions regarding the status and care of the neonate. After each birth two samples of umbilical cord blood are routinely taken for testing ABO blood type and antiglobulin (Coomb's Antibody) to determine baby's blood type and whether or not the maternal immune system has passed any antibodies to the baby. This is important in cases in which the mother is Rh negative and the baby is Rh positive, where treatment of the mother can prevent Rh disease in future pregnancies. Other tests may also be performed on the umbilical cord blood to assess blood gases and pH, blood type and Rh, complete blood count (CBC), platelet count, hemoglobin levels (Hgb), hematocrit (HCT), bilirubin levels, glucose and blood culture (if an infection is suspected), depending on the circumstances.

Existing devices have the risk of an accidental needle stick and exposure to blood-borne diseases such as hepatitis and HIV-AIDS. In the methods of the prior art, the sampling is or may be accomplished by utilizing a hypodermic needle attached to a syringe and drawing off the desired volume of blood directly from the vessels of umbilical cord. Unfortunately, this method has the disadvantage of having the potential of sticking the operator with a bloody needle, or otherwise exposing the operator to blood. Similarly, collection devices that involve collection of blood by gravity into open mouth containers also risk exposing delivery room personnel to blood spills.

### SUMMARY OF THE INVENTION

The system and method of the present invention includes an umbilical cord sampling device comprising a needle assembly having a base and at least one sampling needle operatively linked to a corresponding sampling reservoir. In preferred embodiments, the sampling reservoir is contained in the sampling device or in a removable cassette. The system and method of the present invention provides an enclosed sampling system for avoiding needle stick incidents in a delivery room. The system and method of the present invention collecting samples of umbilical cord fluids without the risk of contamination. In preferred embodiments, the system simplifying sample collection and analysis of umbilical cord fluids providing immediate blood gas and pH information. Additionally, the umbilical sampling device serves to stabilize the umbilical cord segment during sampling, applying pressure to the umbilical arteries and vein, and maintaining the tips of the sampling needles in position in the lumen of the vessels while moving a roller to facilitate drawing blood.

In preferred embodiments, the umbilical cord sampling device includes at least one positionable sampling needle and at least one central sampling needle. In certain embodiments the position of the central sampling needle is fixed in relation to the umbilical cord sampling unit. In some embodiments, a sampling needle is a needle array comprising a hollow needle for withdrawing a fluid sample and a sensor, such as a pH electrode, for measuring a physiologically relevant parameter. In some embodiments, a sensor, such as a pH electrode, is mounted and maneuvered with a positionable sampling needle.

In a preferred embodiment, the top of the needle assembly includes a window or lens. The lens helps the operator adjust positionable sampling needles under visual control, which is useful for penetrating umbilical compartments, such as blood vessels, and sampling the fluid contained therein.

In accordance with a preferred embodiment, the invention provides a method for determining the values of physiologically relevant parameters of a biological fluid, comprising the steps of providing an umbilical cord sampling device having at least one sampling needle operatively connected to at least one sampling reservoir; placing an umbilical cord segment in the umbilical cord sampling device; penetrating a fluid-containing compartment of the umbilical cord segment with a sampling needle; collecting the fluid in a sampling reservoir; and analyzing the collected fluid to determine the values of physiologically relevant parameters. Typically the physiologically relevant parameters include blood pH, blood pO₂ and blood pCO₂. In one embodiment, an aliquot of the fluid sample is withdrawn directly from the sampling reservoirs of the removable cassette into the analysis device. In other embodiments, selected physiological parameters are measured using sensors located within a sampling reservoir of the cassette. After the initial determination of the physiologically relevant parameters in the delivery room, the cassette containing the remaining fluid sample can be transferred to the hospital laboratory for further testing.

In preferred embodiments, the removable cassette mates with a corresponding docking unit that is operatively linked to an analyzer. In preferred embodiments the analytical device provides the ability to determine levels of physiologically relevant blood gases, blood pH and optionally other aspects of blood chemistry. In one embodiment, the docking unit is provided with conduits connected to a docking mating port that functionally mates with a cassette mating port of the removable cassette thereby providing for the withdrawal of fluid samples from sampling reservoirs. In one embodiment, the docking unit provides an actuator that operatively mates with valve and provides the ability to withdraw fluid from a chosen sampling reservoir under automatic control.

In other embodiments, a sample reservoir of the removable cassette includes one or more sensors that measure relevant physiological parameters, such as pH, PO₂, PCO₂, glucose, etc. In such embodiments, a cable and sensor connector operatively linked to the docking unit can mate with corresponding connectors on the cassette, providing sensors for measuring physiological parameters without drawing sample fluid into the docking unit and analyzer, avoiding contamination and reducing required cleaning.

In a preferred embodiment, the removable cassette can be a single use, disposable cartridge for both collecting blood and interfacing with an analyzer in order to perform a variety of critical care assays including, for example, electrolytes, general chemistries, blood gases and hematology. In one embodiment, a cassette available from the i-STAT Corporation of Princeton, N.J. or other manufacturers can be used. When utilizing a removable cassette or cartridge, the docking unit may preferably include a first, sample reservoir that remains within the docking unit, while the removable cassette may preferably include a collection well which can receive blood from the first, sample reservoir, and transport the blood to a corresponding analyzer.

In embodiments in which the collected fluid is blood, analysis of the collected blood is performed using one or more of the following tests: ABO blood type and antiglobulin (Coomb's Antibody) to determine baby's blood type and whether or not the maternal immune system has passed any antibodies to the baby, blood gases and pH, electrolytes, complete blood count (CBC), platelet count, hemoglobin levels (Hgb), hematocrit (HCT), bilirubin levels, glucose, lead, TSH, PKU, toxicology and blood culture (if an infection is suspected), depending on the circumstances. In some embodiments, a sample of blood, or a sample DNA extracted from an aliquot of blood, can be stored for later use, e.g., identification of the patient.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic diagram of an embodiment of the umbilical cord sampling system of the present invention, showing a segment of umbilical cord 10 held in the umbilical sampling device 100, a removable cassette 400 that can be transferred from the umbilical cord sampling device 100 to the docking unit 700 of an analyzer 800 that is in electronic communication with a computer system 900.

FIG. 2 is a schematic diagram of a cross-section of the umbilical cord 10, showing the paired umbilical arteries 20, the central umbilical vein 30, mucous connective tissue (Wharton's jelly) 50 and the amniotic epithelium 60 that covers the umbilical cord.

FIG. 3 is a schematic diagram of the top view of an embodiment of the umbilical cord sampling device 100 with a removable cassette 400 in position.

FIG. 4 is a schematic diagram of a side view of an embodiment of the umbilical cord sampling device 100 with a removable cassette 400 in position.

FIGS. 5A and 5B are schematic diagrams of two embodiments of the umbilical cord sampling device showing a section view in plane A of FIG. 3. FIG. 5A shows an embodiment in which the roller 320 mounted in the base 600 compresses the umbilical cord 10 against the lower surface 230 of the needle assembly 200 of the umbilical cord sampling device. FIG. 5B shows an alternative embodiment in which the roller 320 mounted in the needle assembly 200 compresses the umbilical cord 10 against the upper surface 620 of the base 600 of the umbilical cord sampling device.

FIG. 6 is a schematic diagram of the top view of a portion of an embodiment of the umbilical cord sampling device, without a removable cassette in position, showing details of the meter 330 and positionable sampling needles 520 and 540.

FIG. 7 is a schematic diagram of the top view of an embodiment of a removable cassette showing sensors 490 in the sampling reservoir 410 and a probe 484 that are operatively connected directly through a plug 496 and cable 498 to the docking unit 700.

FIG. 8 is a schematic diagram illustrating an embodiment of a needle housing mating port 290 and an embodiment of a removable cassette mating port 480.

FIG. 9 is a schematic diagram illustrating a removable cassette 400, a docking unit 700, and an analyzer 800 operatively linked to a computer system 900.

FIG. 10 is a schematic diagram illustrating a method for determining the values of physiologically relevant parameters of a biological fluid, comprising the steps of providing an umbilical cord sampling device having at least one sampling needle operatively connected to at least one sampling reservoir; placing an umbilical cord segment in the umbilical cord sampling device; penetrating a fluid-containing compartment of the umbilical cord segment with a sampling needle; collecting the fluid in a sampling reservoir; and analyzing the collected fluid to determine the values of physiologically relevant parameters.

FIG. 11 is a perspective view of a second embodiment of the umbilical cord sampling device of the present invention.

FIG. 12 is an enlarged, perspective view of the embodiment of FIG. 11 showing insertion of a syringe for removal of fluid from an internal reservoir.

FIG. 13 is an end view, in partial cross-section of the umbilical cord sampling device of the embodiment of FIG. 11.

FIG. 14 is a side view of the umbilical cord sampling device of FIG. 11.

FIG. 15 is a side view in partial cross section of the umbilical cord sampling device of FIG. 11 showing insertion of a removable cassette.

FIG. 16 is a partial perspective view of the umbilical cord sampling device showing insertion of the cassette of FIG. 15.

FIG. 17 is an end view, in partial cross-section of the umbilical cord sampling device of the embodiment of FIG. 15 prior to insertion of the cassette.

FIG. 18 is an end view, in partial cross-section of the umbilical cord sampling device of the embodiment of FIG. 15 upon insertion of the cassette.

FIG. 19 is an end view of the embodiment of FIG. 11 in a closed position.

FIG. 20 is an end view of the embodiment of FIG. 11 in an open position.

FIG. 21 is a top view of the of an exemplary cassette.

FIG. 22 is an exploded view of the cassette of FIG. 21.

### DETAILED DESCRIPTION OF THE INVENTION

In general, “needle” or “sampling needle” is used herein to refer to a sharp elongate structure used to penetrate a compartment within a segment of the umbilical cord. The needle comprises a stiff component constructed from metal, glass, suitable polymers or combinations thereof.

In one set of embodiments, “needle” or “sampling needle” is used herein to refer to hollow needles, such as hypodermic needles, that are used to collect fluid samples from the umbilical compartments, preferably from the umbilical arteries or the umbilical veins. Typically a hollow needle has a sharpened beveled tip on the distal end that contacts the umbilical cord. Typically the end opposite to the sharpened beveled tip is connected by a sample conduit or a sample channel or a combination thereof, directly or indirectly through an optional interposed valve, to a sample reservoir. The gauge of the sampling needles is chosen to accommodate the diameter of the umbilical blood vessels.

In another set of embodiments, “needle” or “sampling needle” is used herein to refer to sensors that can be placed in the tissue or fluid-filled compartments of the umbilical cord segment. Such sensors can measure physical parameters such as temperature or chemical parameters such as the presence or concentration of an analyte. In a preferred embodiment, the needle is a pH electrode and the umbilical cord sampling device further comprises a reference electrode. Suitable pH electrodes are known, such as those disclosed in U.S. Pat. No. 6,567,679. In preferred embodiments, the sampling needle is an array comprising a hollow needle and at least one sensor, such as a pH electrode, joined to the hollow needle to allow both fluid sampling and monitoring of a physiologically relevant parameter by simultaneous penetration of a fluid filled compartment.

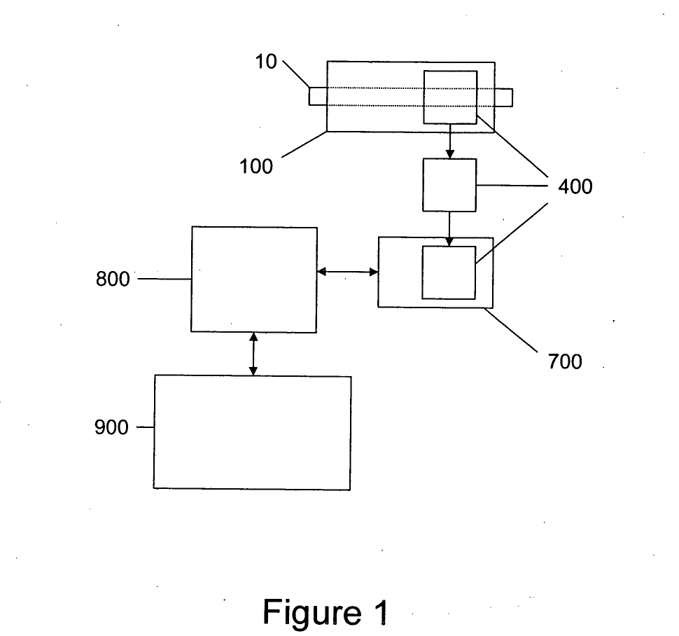

FIG. 1 is a schematic diagram of an embodiment of the umbilical cord sampling system of the present invention, showing a segment of an umbilical cord 10 held in umbilical sampling device 100, a removable cassette 400 that can be transferred from the umbilical cord sampling device 100 to the docking unit 700 of an analyzer 800 that is in operative communication with a networked computer system 900. The operative communication can be by wired or wireless connections. In preferred embodiments the removable cassette has no exposed needles, thereby minimizing the risk of needle-stick incidents during sampling and transfer.

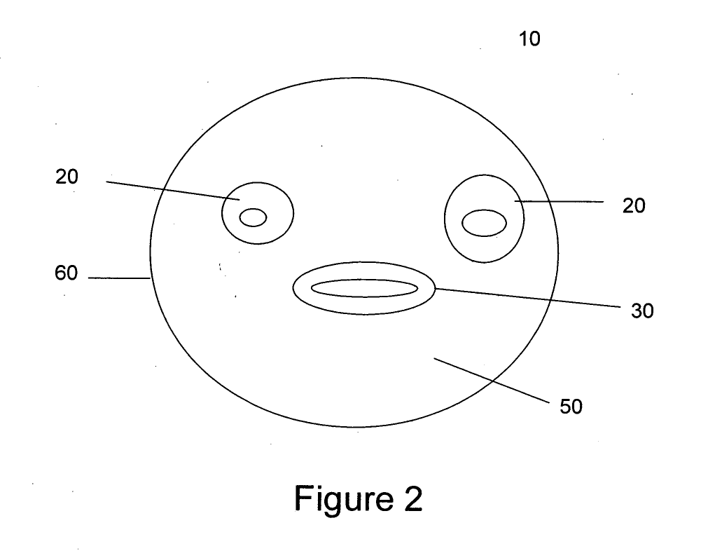

FIG. 2 is a schematic diagram of a cross-section of the umbilical cord 10, showing the paired umbilical arteries 20, the central umbilical vein 30, mucous connective tissue (Wharton's jelly) 50 and the amniotic epithelium 60 that covers the umbilical cord. The umbilical cord is about 1-1.5 cm in diameter, the umbilical arteries are about 0.3-0.4 cm in diameter and the umbilical vein is about 0.6-0.8 cm in diameter.

The needle assembly and the base of the umbilical cord sampling device are secured together by one or more latches or connectors. In a preferred embodiment, the latches have releasable connectors to provide for convenient use in the delivery room environment. In preferred embodiments, the needle assembly comprises a needle housing, a removable cassette including at least one sample reservoir, and a sampling needle. In preferred embodiments, the needle assembly further comprises a meter. The base can comprise a roller assembly and a base housing.

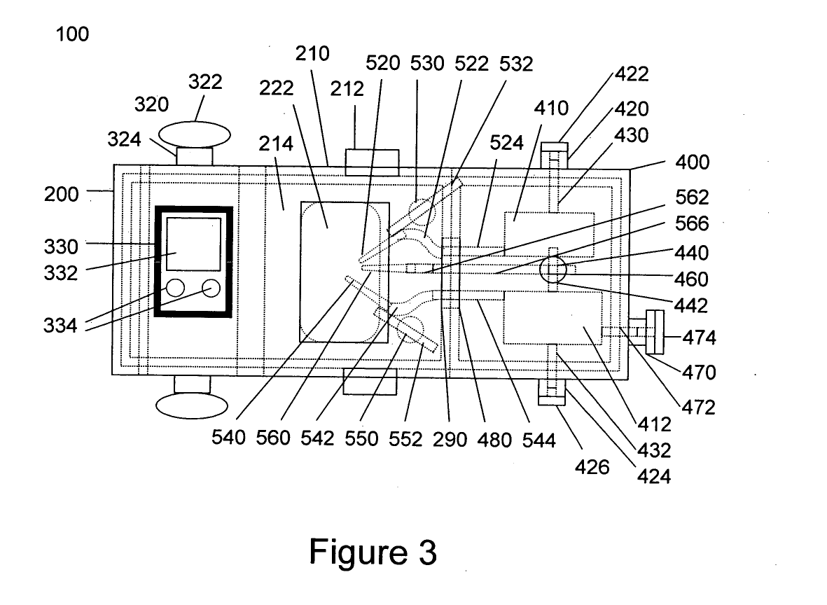

FIG. 3 is a schematic diagram of the top view of an embodiment of the umbilical cord sampling device 100 showing the top of the needle assembly 200 with a removable cassette 400 in position, and a roller assembly including roller 320, roller knobs 322 and roller shaft 324. In preferred embodiments, the needle assembly 200 comprises an needle housing 210, including a positionable sampling needle assembly, and a removable cassette 400. In preferred embodiments, the needle assembly 200 further comprises a meter 330.

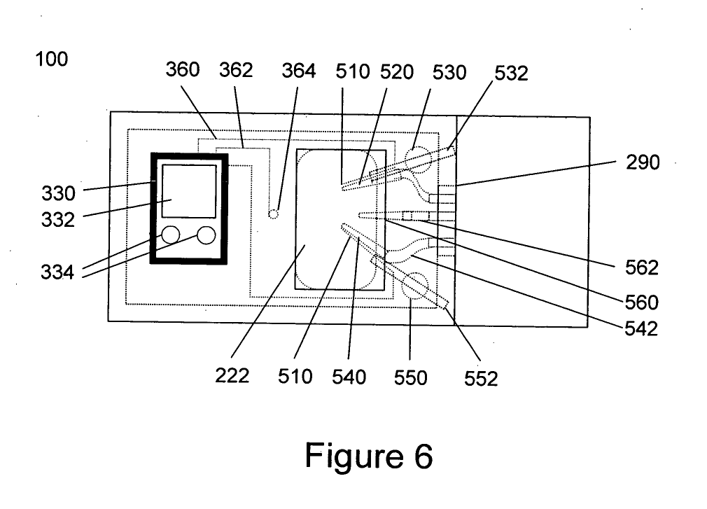

The meter 330 comprises a display 332 and controls 334. Further details of meter connections are shown in FIG. 6. The meter provides an immediate read-out of a physiologically relevant parameter, such as pH. One control can be used to activate or reset the parameter reading. In preferred embodiments, the meter also includes a clock function, including the ability to run a timer that can be started at the time of birth.

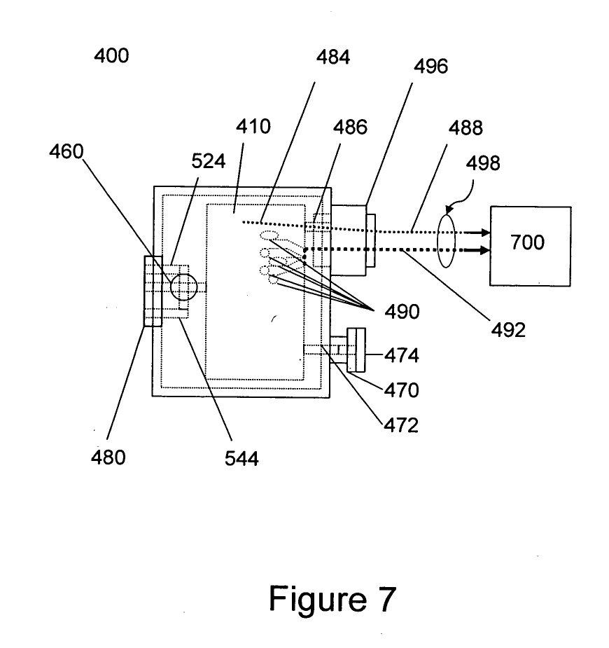

The removable cassette comprises at least one sample reservoir. In a preferred embodiment, the removable cassette comprises a first sample reservoir 410 connected by a first test channel 430 to a first test port 420 having an elastomeric septum 422. An optional second sample reservoir 412 is connected by a second test channel 432 to a second test port 424 that has an elastomeric septum 426. The first connecting channel 440 leads from the first sample reservoir 410 to a valve 460. Similarly, the second connecting channel 442 leads from the second sample reservoir 412 to the valve 460. In a preferred embodiment, a central sampling needle channel 566 leads from cassette mating port 480, needle housing mating port 290, and the central sampling needle conduit 562 to valve 460. The valve 460 can be adjusted to block flow from the central sampling needle channel 566 or to direct fluid from the central sampling needle channel 566 to either the first sample reservoir 410 or the second sample reservoir 412. A syringe port 470, preferably having a standard “Luer-Lok™” connection and elastomeric septum 474, is connected to a sample reservoir (in this embodiment the second sample reservoir 412) by the syringe port channel 472. An alternative embodiment of removable cassette 400 is shown in FIG. 7, below.

In the embodiment depicted in FIG. 3, the first positionable sampling needle 520 and the second positionable sampling needle 540 are visible through a window or a lens 222. Each of positionable needles are directed under visual control to an umbilical vessel using the corresponding positionable sampling needle handle 532, 552. The positionable sampling needles 520, 540 are enclosed within the needle assembly housing 210 and affixed to the ends of the handles 532, 552. The handles 532, 552 and mounts 530, 550 of the respective positionable sampling needles 520, 540 provide the operator with control of the position of the tip of the positionable sampling needles in preferably at least two dimensions. In a preferred embodiment, the mount is a ball freely movable in a socket formed in the upper surface of the needle assembly and the handle is a joystick that passes through the ball such that the sampling needles may be moved in three dimensions.

In a preferred embodiment, a central sampling needle 560 is fixed with respect to the needle housing 210. Alternatively, the central sampling needle 560 can be positionable with respect to the needle housing 210.

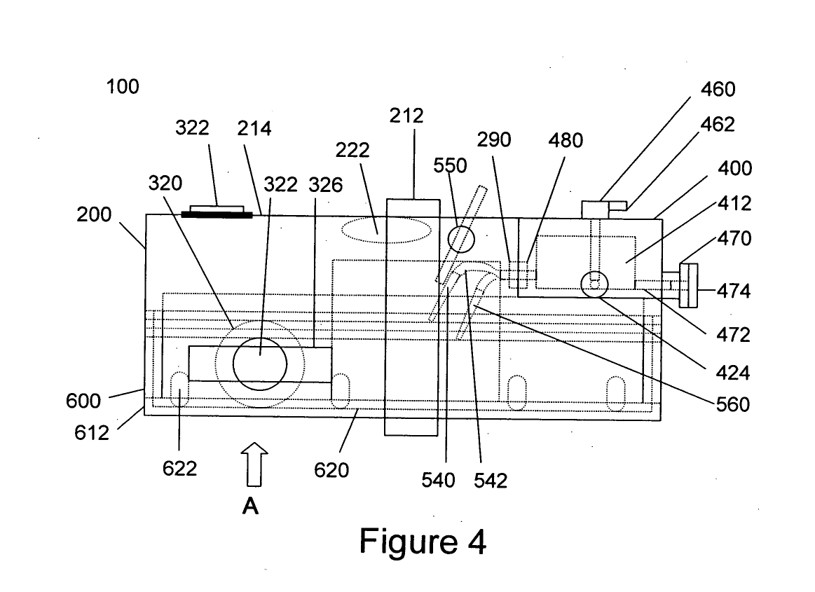

FIG. 4 is a schematic diagram of a side view of an embodiment of the umbilical cord sampling device 100 with a removable cassette 400 in position.

The needle housing has a top 214 having an upper surface and a lower surface, a first end wall, a second end wall, a first lateral wall having a needle housing locking edge and a second lateral wall having a needle housing locking edge. Two latches 212 are shown that are used to connect the needle assembly 200 and the base 600. The lens 222 can be a simple lens or a compound lens and can be made of any suitable material, preferably an optically suitable plastic such as a polycarbonate. In a preferred embodiment, the lens 222 is molded into the top of the needle assembly. The lens 222 has focal length and power, preferably 1.5-2.5×, chosen to image the surface of the umbilical cord segment to facilitate impaling blood vessels under visual control with a positionable sampling needle 540. Alternatively, a window may be provided, without any magnification capabilities, but which allows viewing of the needle assembly during use.

The base housing has a bottom having an upper surface 620 and a lower surface, a first end wall, a second end wall, a first lateral wall having a lower housing locking edge and a second lateral wall having a lower housing locking edge. The cord receiver of the base is the space defined by the upper surface of the bottom, the first end wall, the second end wall, the first lateral wall and the second lateral wall, that serves to contain a segment of umbilical cord when the umbilical cord sampling device is in use. Several cord support blocks 622 are affixed to the upper surface 620 of the bottom 612, and extend into the space of the cord receiver. The profile of the cord support blocks 622 is adapted to support and immobilize the segment of umbilical cord during the sample procedure.

As shown in FIG. 3, the roller assembly includes a roller 320 and a roller shaft that has a roller knob 330 at each end. In preferred embodiments, the roller shaft travels in a roller track 326 that is defined by a slot in each of the lateral walls of the base 600. In other embodiments, the roller shaft travels in a roller track that is defined by a slot in each of the lateral walls of the needle assembly 200.

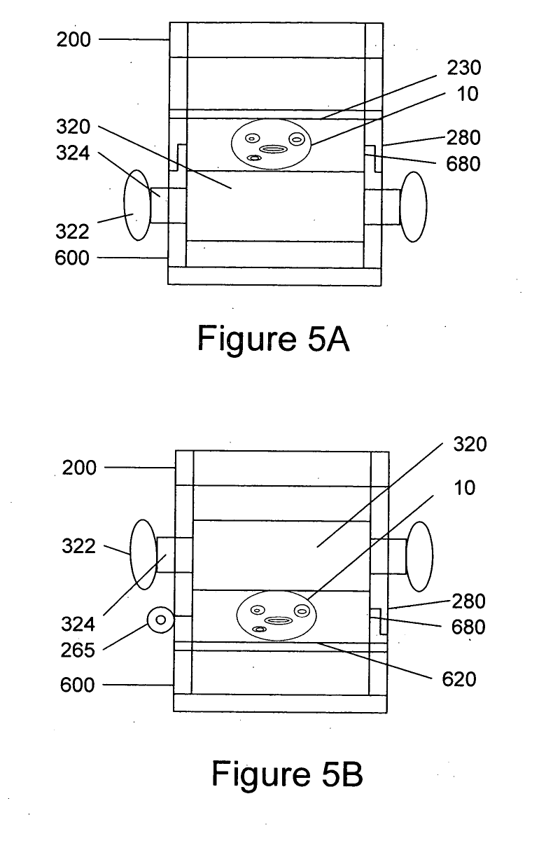

FIGS. 5A and 5B are schematic diagrams of two embodiments of the umbilical cord sampling device showing a section view in plane A of FIG. 4. FIG. 5A shows an embodiment in which the roller 320 mounted in the base 600 compresses the umbilical cord 10 against the lower surface 230 of the needle assembly 200 of the umbilical cord sampling device. FIG. 5B shows an alternative embodiment in which the roller 320 mounted in the needle assembly 200 compresses the umbilical cord 12 against the upper surface 620 of the base 600 of the umbilical cord sampling device. FIGS. 5A and 5B also illustrate the upper housing locking edge 280 and the lower housing locking edge 680 that serve to stabilize the needle assembly 200 with respect to the base 600.

As shown in FIG. 5B, one or more hinges 265 can be placed on the first lateral wall of the base and pivotably linked to corresponding hinges 265 on the first lateral wall of the needle assembly. In one embodiment the hinges are “live” hinges made of a flexible material. In another embodiment, the hinges are linked by one or more hinge pins. The relative position of the needle assembly and base is stabilized by locking edges 280, 480, one or more latches (212 in FIGS. 2 and 3). The hinges 265 can extend the entire length of the umbilical sampling device or only one or more segments of the length.

A segment of umbilical cord is provided by conventional means. In one embodiment, a first and second clamp are placed pairwise on the cord towards the newborn. The clamps may be specialized umbilical cord clamps, but other clamps, such as hemostats or Kelly clamps can be used. The amount of blood and other fluids in the cord segment can be increased by manually “milking” from the placental side towards the first and second clamps. The third and fourth clamps are applied about 10-15 cm towards the placenta from the first and second clamps. The cord segment is cut between the first and second and between the third and fourth clamps. The umbilical cord segment and attached clamps is placed into the umbilical cord sampling device. Alternatively, other techniques and approaches that produce a clamped 10-15 cm umbilical cord segment may be used.

In a preferred embodiment, the umbilical cord sampling device 100 is assembled for use by placing a segment of umbilical cord into the cord receiver of the base; aligning the locking edge 680 of the base to the locking edge 280 of the needle assembly; applying sufficient pressure to join the base to the needle assembly by interlocking the respective locking edges, and stabilizing the joined base and needle assembly using at least one latch 212. As described above, the segment of umbilical cord is clamped at both ends. Preferably, the clamped ends of the umbilical cord segment extend beyond the end walls of the cord receiver. In a preferred embodiment, the end walls of the assembled umbilical cord sampling device 100 (respectively, the first end wall of the needle housing 240 and the first end wall of the base housing 640; and the second end wall of the needle housing 250 and the second end wall of the base housing, 650) are disposed to immobilize the clamped cord segment.

In placing the umbilical cord into the cord receiver of the base, the umbilical cord segment is aligned so that the central sampling needle 560 is positioned to penetrate the central umbilical vein 30. The ends of the umbilical cord segment that extend beyond the end walls of the cord receiver are conveniently manipulated while aligning and joining the needle assembly and base to penetrate the central umbilical vein 30 with the central sampling needle 560.

Once the needle assembly and base have been attached, positionable sampling needles can be used to penetrate an umbilical compartment, preferably one or both umbilical arteries 20. The first positionable sampling needle 520 and the second positionable sampling needle 540 are visible through the lens 222. The positionable needles are directed under visual control to an umbilical vessel using the corresponding positionable sampling needle handle 532, 552. The handle and mount of the positionable sampling needles provide control of the position of the tip of the positionable sampling needles in three dimensions. After the positionable sampling needle is maneuvered over the umbilical vessel, it is advanced into the vessel by pushing on the handle. Blood is withdrawn into a corresponding sample reservoir via the respective sample conduit and sample channel.

The flow of blood into the positionable sampling needles can be. facilitated by establishing a pressure gradient from the lumen of the blood vessel to the sampling reservoir. This can be done by several methods individually or in combination. Positive pressure can be applied to the blood in the vessels using the roller 330. Alternatively, the sample reservoirs can be under a slight negative pressure that is maintained by elastomeric septa (482, 422, 426 and 472) that seal the openings of cassette mating port, first test port, second test port and syringe port, respectively. Alternatively, negative pressure can be applied using a syringe operatively mated to syringe port 470.

In preferred embodiments, sample reservoirs of the cassette are heparitiized by coating the inner surfaces with a Group 1 or Group 2 metal salt of heparin, preferably selected from the group consisting of lithium heparin, sodium heparin, magnesium heparin, and calcium heparin. In preferred embodiments lithium heparin is used.

When used, the roller 320 compresses the umbilical cord segment against an opposing surface. Blood within the umbilical vessels is peristaltically “milked” toward the sampling needles by movement of the roller shaft.

FIG. 6 is a schematic diagram of the top view of a portion of an embodiment of the umbilical cord sampling device 100 without a removable cassette in position, showing details of the meter 330 and positionable sampling needles 520 and 540. In the illustrated embodiment, the first sampling needle 520 includes a sensor 510, such as a pH electrode, that is operatively connected to the input of meter 330 by a sensor conductor 360. The sensor may be mounted alone to first sampling needle handle 532, or may be mounted as a component of an array including a hollow needle for sampling fluid, as shown in FIG. 6. A reference electrode 364 is placed on the lower surface of the bottom of the needle assembly where it makes electrical contact with the umbilical cord segment. The reference electrode 364 is connected to the input of meter 330 by reference electrode conductor 362. Meter 330 has a display 332 and controls 334.

FIG. 7 is a schematic diagram illustrating an embodiment of a removable cassette 400 having a single first sample reservoir 410 and the valve 460 interposed between the cassette mating port 480 and the first sample reservoir 410. In this embodiment a probe 484 enters through a probe port 486 to measure physiological parameters such as pH. In addition, one or more sensors 490 are positioned in contact with the fluid within the first sample reservoir 410. In preferred embodiments, at least one sensor is a thermal probe. In preferred embodiments, a sensor array of more than one sensor 490 is present within the first sample reservoir 410. In preferred embodiments, the sensors in a sensor array are affixed to a common substrate.

Arrays of sensors suitable for measuring relevant physiological parameters are known. See, for example, Lauts, I.R., Microfabricated biosensors and microanalytical systems for Blood Analysis, Accounts of Chemical Research 1998, 31 (5):317-324 and references cited therein, which are incorporated by reference in their entirety. Conductors providing electrical signals from the sensors 490 are present in a cable 498 that is functionally connected to the docking unit 700. One or more probe conductors 488 connecting respective probes 484 and 490 to the docking unit 700 also pass through the cable 498.

In preferred embodiment connection between the sample cassette 400 and the docking unit 700 are made using a sensor connector 496, which terminates the cassette end of cable 498. Also diagrammatically illustrated in FIG. 7 are the cassette mating port 480, first positionable sampling needle channel 524, second positionable sampling needle channel 544, syringe port 470, syringe port channel 472 and syringe port septum 474.

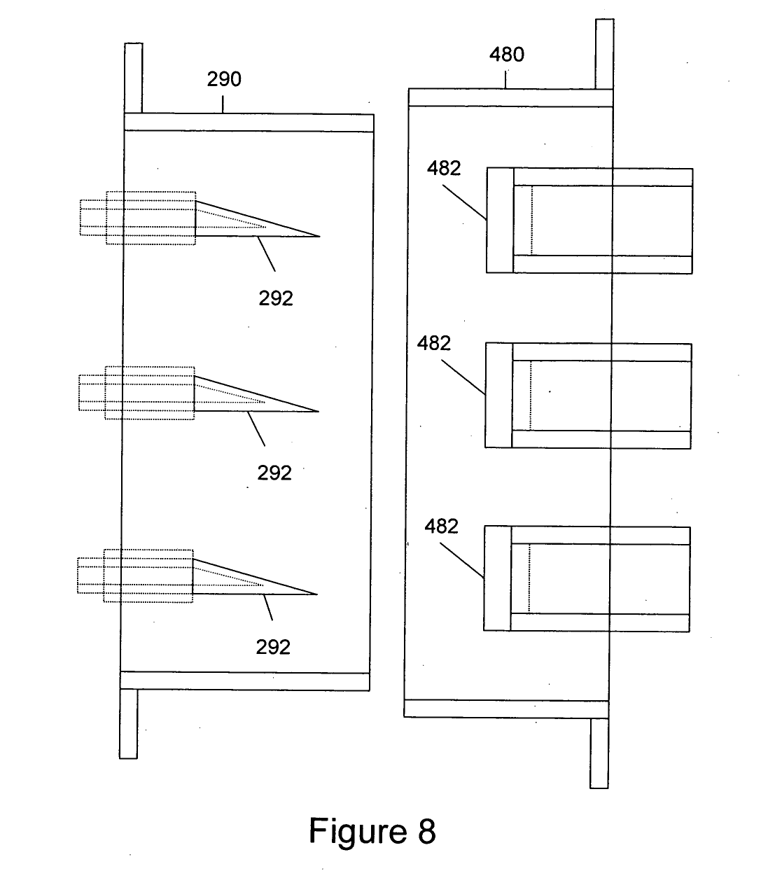

FIG. 8 is a schematic diagram illustrating an embodiment of a needle housing mating port 290 and an embodiment of a corresponding cassette mating port 480. In preferred embodiments, there are no exposed needle on the surface of the cassette mating port 480. In preferred embodiments, any sharp needle tips 292 are recessed. The docking unit mating port is identical to the needle housing mating port 290. The openings of the cassette mating port 480 are sealed by elastomeric septa 482. When the septa 482 are pierced by needle tips 292, communication is established via mating ports 290 and 480 between first sampling needle conduit and first sampling needle channel, central sampling needle conduit and central sampling needle channel, and between second sampling needle conduit and second sampling needle channel, respectively when the cassettes is placed in the needle assembly or removed and placed in the docking unit.

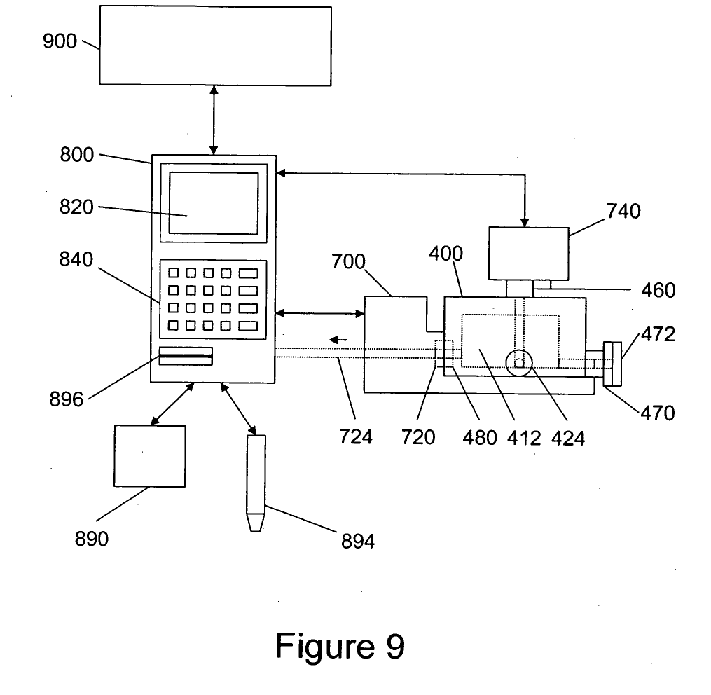

FIG. 9 is a schematic diagram illustrating the umbilical cord sampling system, showing a corresponding cassette 400, a docking unit 700, an analyzer 800 and a handheld computer system 900. After the sample has been drawn into the cassette 400 the cassette 400 is detached from the umbilical cord sampling device (100, FIG. 4) and placed in the docking unit 700 .of the analyzer 800. The septum of each opening of the cassette mating port 480 closes on removal from the needle housing mating port (290, FIG. 4) of the umbilical cord sampling device, thereby preventing contamination of the sample and possible contamination of the surroundings by leakage of possibly infected fluids. Each septum is re-opened by insertion into the corresponding docking unit mating port 720. In one embodiment, samples can be withdrawn from the second sample reservoir 412, the first sample reservoir (410, FIG. 3) or from both. In some embodiments the valve 460 can be rotated by an actuator 740 under the control of the analyzer 800, if required. The first test port septum (422, FIG. 3), the second test port septum (426, FIG. 3) or the syringe port septum (472, FIG. 3) can be removed or punctured to equalize pressure and facilitate sample removal through the cassette mating port 480. Alternatively, sample fluids can be removed directly through first text port (420, FIG. 3), the second text port (424, FIG. 3) or the syringe port (470, FIG. 3) whether or not the cassette 400 is placed on the docking unit 700. In a needle-less procedure, the septum can be removed from a test port and a sample withdrawn with a capillary. If necessary, such removal of a sample can be facilitated by application of positive pressure using a syringe attached to the syringe port.

A suitable analyzer 800 provides the ability to determine the value of at least one of blood pH, blood PO₂ and blood pCO₂. In general, blood gas analysis involves the direct measurement of pH, PO₂, and pCO₂ and can include the following calculated parameters: HCO₃⁻, standard bicarbonate (SB), buffer base (BB), base excess (BE), base excess extracellular fluid (BEecf), percentO₂ saturation (SO₂), O₂ content (ctO₂), and total CO₂ concentration (ctCO₂). Existing blood gas analyzers use three types of electrode systems to determine pH, pCO₂, and pO₂ in the blood.

In preferred embodiments the analyzer 800 is equipped with a display 820, a keypad 840, and operative connections to a printer 890 and a bar code reader 894. In some embodiments, the analyzer 800 is equipped with an electronic card reader 896 that can be integrated into the analyzer or located in a separate housing. The analyzer 800 is operatively connected to the docking station 700 by physical connections, infrared link or wireless link. Optionally in embodiments in which fluids are analyzed within the analyzer, the analyzer and the docking unit are connected by a fluid channel 724. In embodiments in which analysis of the sample fluid is performed within the cassette or within the docking unit, the analyzer and the docking unit are operatively linked by a direct physical connection, infrared link or wireless link. In some embodiments, the analyzer and the docking unit are integrated into a single device. In some embodiments, the analyzer 800 is a hand-held device comprising a microprocessor, for example, a PDA, Pocket PC or handheld computer.

In preferred embodiments the analyzer 800 and the docking unit 700 are positioned on a counter or table in a non-sterile area of the operating room. A circulating nurse can receive the cassette 400 from a scrub nurse or physician, place the cassette 400 in the docking unit 700 and read the results of analysis from the display 820. A paper copy of the results is provided by the printer 890.

In preferred embodiments, the extraction of sample fluid, analysis of the physiological parameters such as blood pH, blood PO₂ and blood pCO₂ are automatically controlled by the analyzer 800 by the execution of a stored program. The program can be initiated by the detection of a cassette 400 placed in the docking unit 700. Alternatively, the program can be initiated by instructions entered by an operator using the keypad 840, bar code reader 894 or electronic card reader 896. Additional information, such as patient identifier and time of birth, can be entered at the analyzer and transmitted with the analysis results to the central computer system 900 to be stored in the database of patient information.

In preferred embodiments, communications between docking unit 700 and analyzer 800 and between analyzer 800 and the computer system 900 conform to relevant industry standards such as Health Level Seven (HL7), IEEE1073 (ISO 11073) and IEEE 802. The computer system can be a standard desktop system with local memory or can be connected to a central hospital server to access a patient database located remotely. Operative communication links can be wired or wireless.

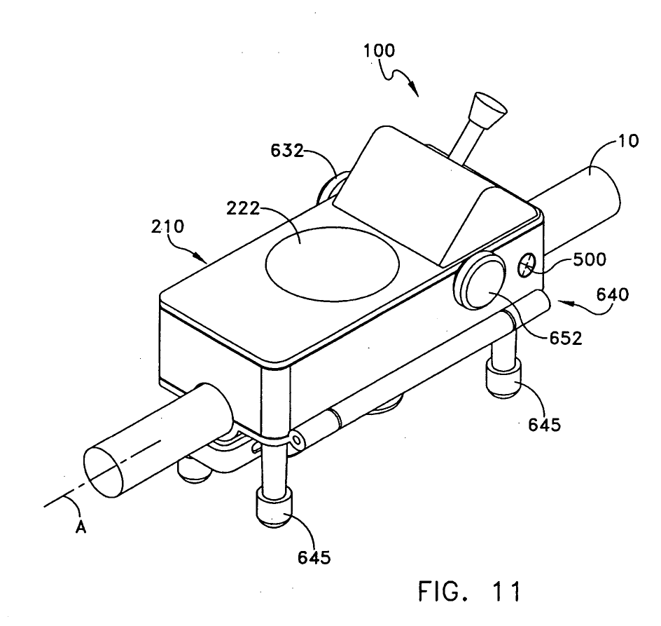

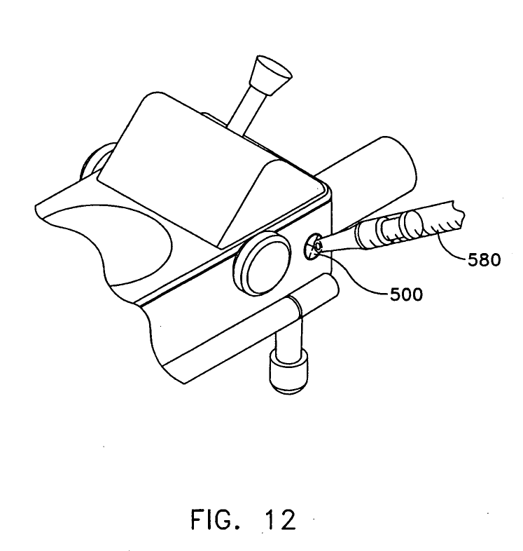

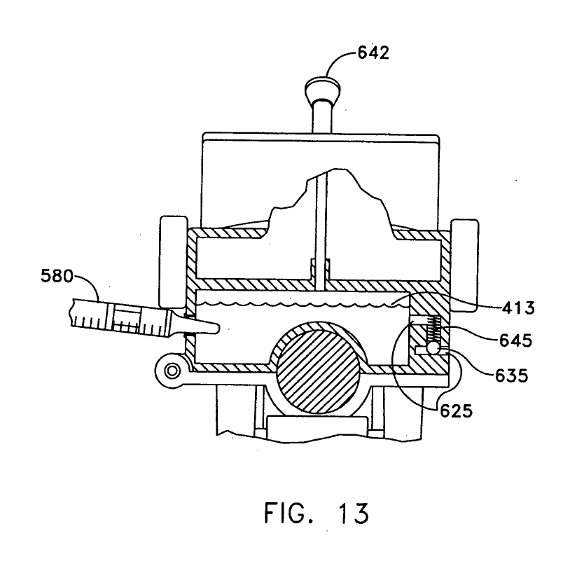

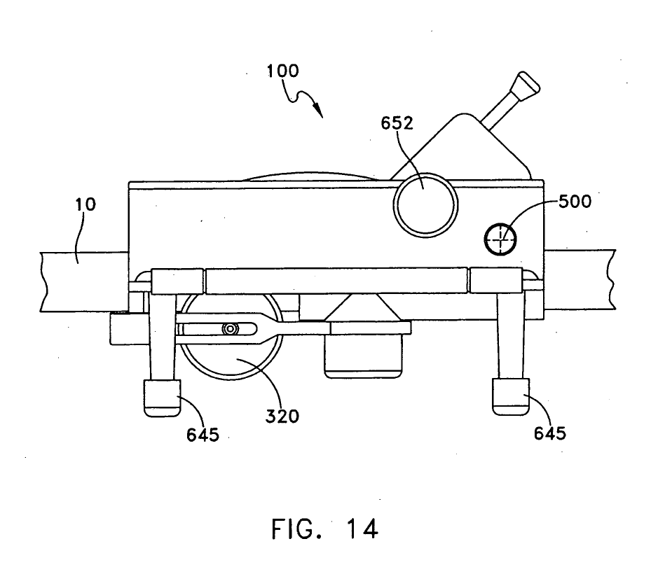

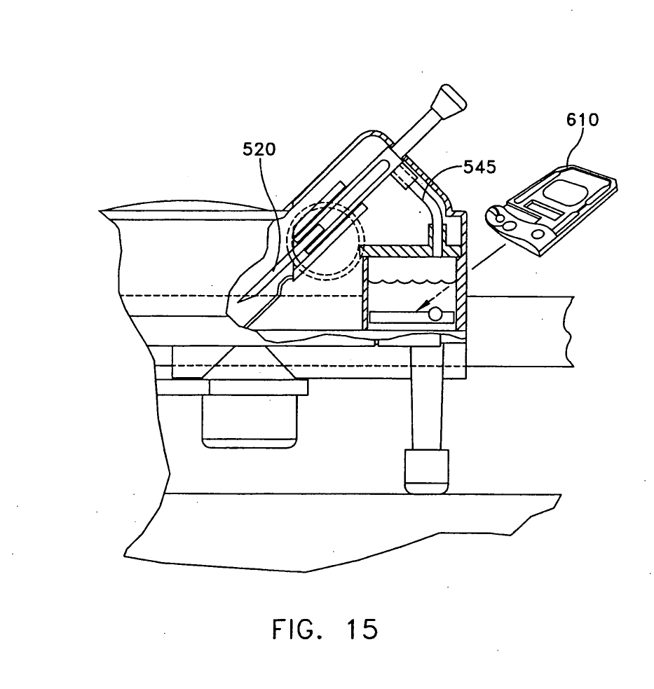

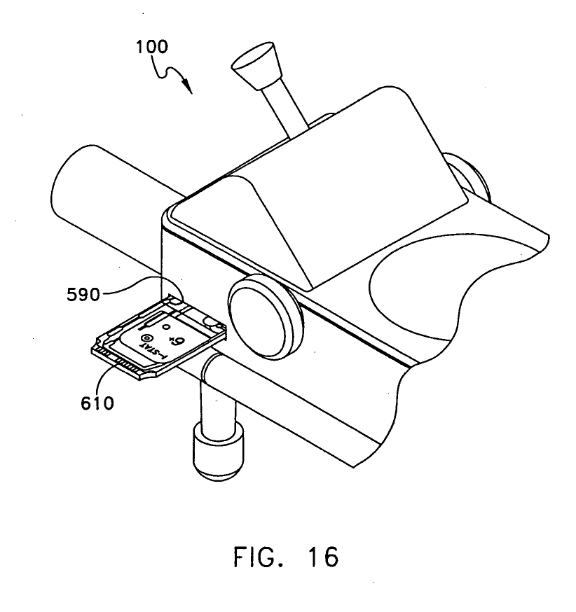

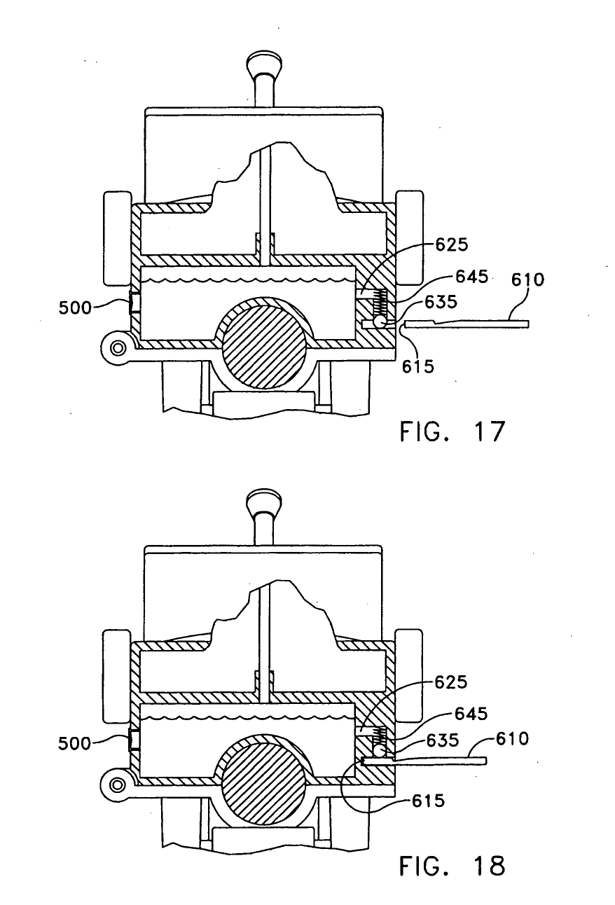

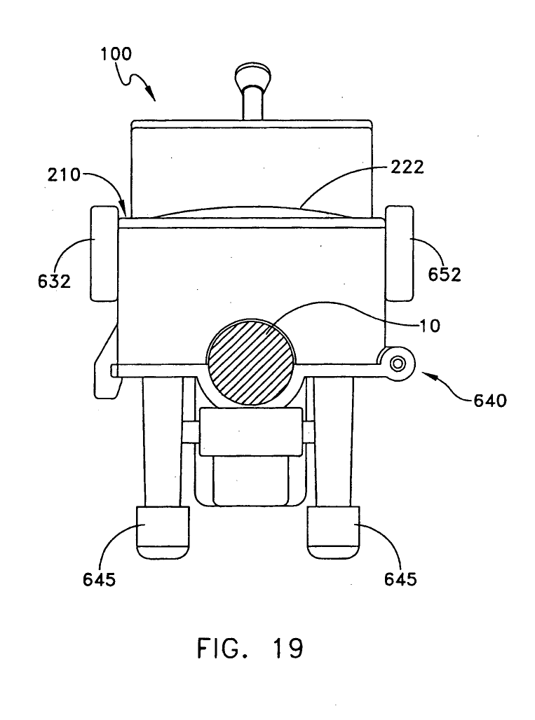

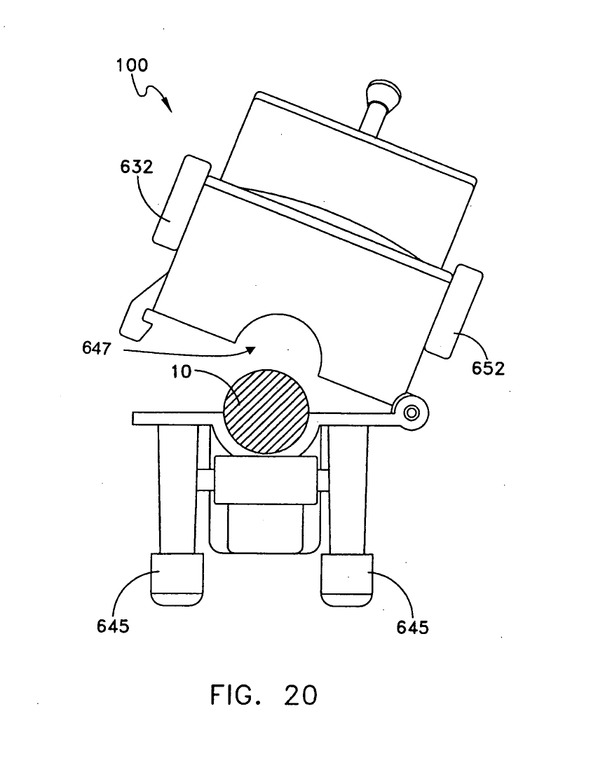

Referring now to FIGS. 11-22 another embodiment of the umbilical cord sampling device is illustrated which includes a sampling reservoir supported within the base. In this embodiment, members which are the same or similar to those in the previous embodiments are numbered the same. The umbilical cord sampling device 100 of the present embodiment includes a needle housing 210 and a base housing 640 which are preferably moveable between an open (FIG. 20) and a closed position (FIG. 19) in order to position and support a segment of umbilical cord 10 in a cavity 647, as disclosed above. In the present embodiment, legs 645 are provided on each of the comers of the base housing in order to provide support to the device when placed on a surface, for example a hospital table. The legs may be adjustable or foldable, if desired. The device may also preferably include one or more needles, for example, a first positionable sampling needle 520 and a second positionable sampling needle 540 that are visible through a window or a lens 222 during use. Each of positionable needles are directed under visual control to an umbilical vessel using the corresponding positionable sampling needle handle or knobs 632,652. In the present embodiment, the knobs 632, 652 move the first and second sampling needles 520, 540 transverse with respect to an axis “A” of the umbilical cord. Once positioned, a plunger 642 is used to move the first and second sampling needles into the umbilical cord. A roller 320 may also be provided in a roller track 326 disposed along a bottom of the base housing in order to compress or “milk” the umbilical cord, as also described above.

The sampling reservoir 413 is preferably in fluid communication with positionable sampling needles 520, 540, via a conduit 545 as described above, and is preferably accessible from the exterior by a syringe, needle, capillary tube, and/or a removable cassette or the like. In the present embodiment, a port 500 is provided which is supported on the body of the device, for example through a side wall of the needle housing, and which is in fluid communication with the reservoir 413. The port provides access to the reservoir in order to remove a sample, for example via syringe 580. The sampling reservoir 413 may also be accessed through a second port or opening by a removable cassette 610 for example a single use, disposable cassette or cartridge that collects and stores the blood for testing and which may also house pertinent test reagents. In the present embodiment, a slot 590 is disposed through the side wall of the needle housing, for example on the side opposite the port 500. The slot is configured and dimensioned to fit a sampling end 615 of the cassette 610 therein. To access the fluid within the reservoir 413, a valve 635 is provided which is in fluid communication with the interior of the reservoir. The valve 635 is preferably biased in a closed position in order to prevent excess fluid leakage through the valve, especially absent the presence of a cassette. In the present embodiment, a conduit 625 having a ball valve 635 is illustrated, the ball being biased by a spring 645 into a closed position. Alternatively, other types of valves and biasing devices may be utilized, for example an elastomeric valve, as would be known to those of skill in the art. The ball valve may preferably be sized for engagement with a collection well 655 or other opening disposed in cassette 610 for collection of fluid. When the cassette is inserted within the slot the sampling end 615 engages the ball valve 635 in order to move the ball upward, against the biasing force of the spring 645. Once the ball is moved upward (FIG. 18), fluid is free to flow through conduit 625 and into the opening on the cassette. After the cassette is removed, the ball valve is biased by the spring into the closed position to cut off the flow of fluid. A second cassette may then be inserted and the procedure repeated. Since each cassette requires only about 2-3 drops of fluid for analysis, multiple cassettes may readily be utilized with a single reservoir. Alternately, syringe 580 may be inserted through port 500 in order to remove a sample of fluid from the reservoir.

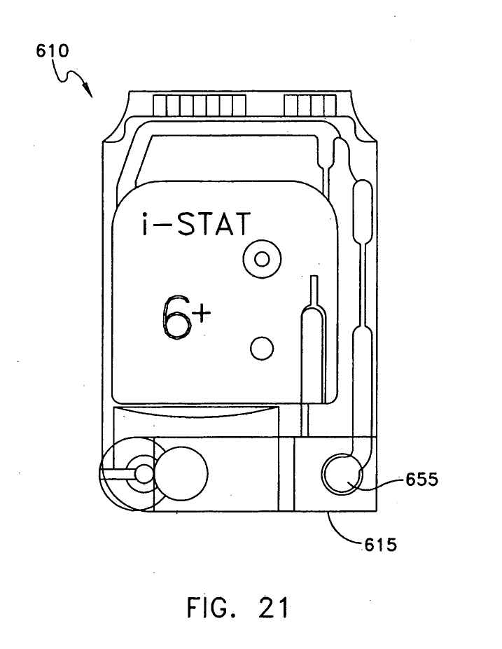

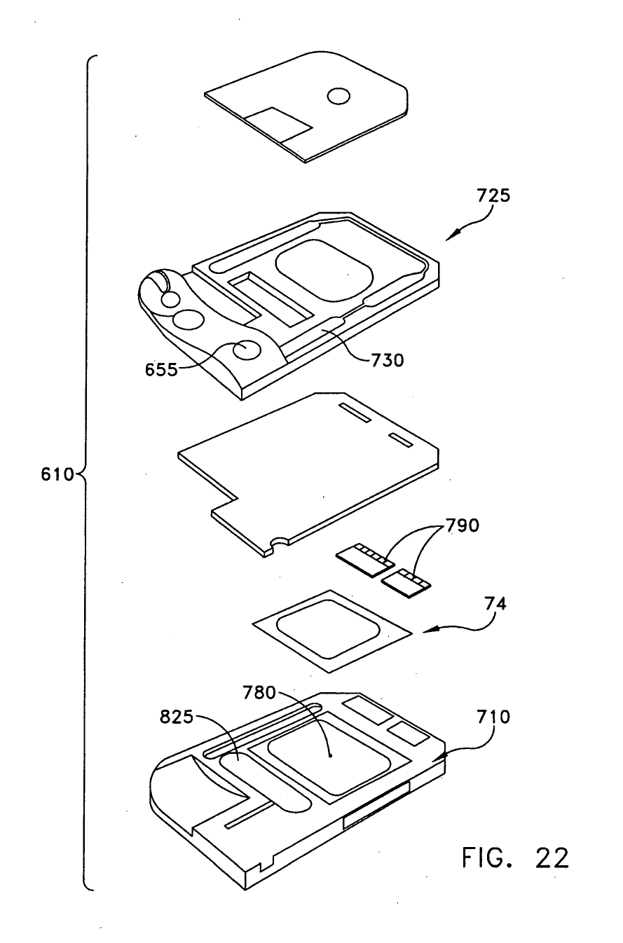

One type of cassette 610 which may be utilized with the present embodiment is a point of care cassette manufactured by the i-STAT Corporation of Princeton, N.J. The i-STAT® cassette, an exemplary embodiment of which is illustrated in FIGS. 21-22, includes a base 710 and a cover 725. The cover has an opening or a collection well 650 for receiving a sample of the fluid, for example blood, therein. In the present embodiment, the ball valve 635 is sized such that when the sampling end 615 of the cassette is inserted within slot 590 the wall around the well engages the ball to push it upward into the open position, as described above. The collection well 655 is in fluid communication with a channel 730 that carries the fluid into the enclosed body of the cassette for storage and testing. Because the cassette is enclosed, it may be handled without the blood spilling or contacting the user. The i-STAT cassettes are each designed to be utilized to perform a particular test or set of tests. Each cassette includes a calibrant solution which is contained within a pouch 745 that is supported within the base 710 of the cassette. The calibrant is preferably a pH buffered aqueous solution of analytes at known concentrations for performing the particular test, for example glucose testing. A sharp member or barb 780 is supported within the base of the cassette, out of contact with the pouch 745 during collection, but is pressed into the pouch when the cassette is placed within a corresponding analyzer, which is manufactured to be utilized with the disposable cassettes. As the barb punctures the pouch, the calibrant fluid is released and follows a flow path that leads it over a sensor array, for example biosensor chips 790, for measurement. When calibration is complete, an air bladder 825 is compressed, for example by operation of the analyzer, so that the calibrant is evacuated into a waste reservoir. This action also causes the blood sample which is supported within the cassette to flow over the sensor array for testing. Such testing may include, for example determining the value of at least one of blood pH, blood pO₂ and blood pCO₂,as described above. Such point of care devices are becoming increasingly popular and although shown and described with an i-STAT cassette, any suitable point of care cartridge and analyzer may be utilized and the slot may be readily adapted according to the shape and size of such a cassette, as would be known to one of skill in the art.

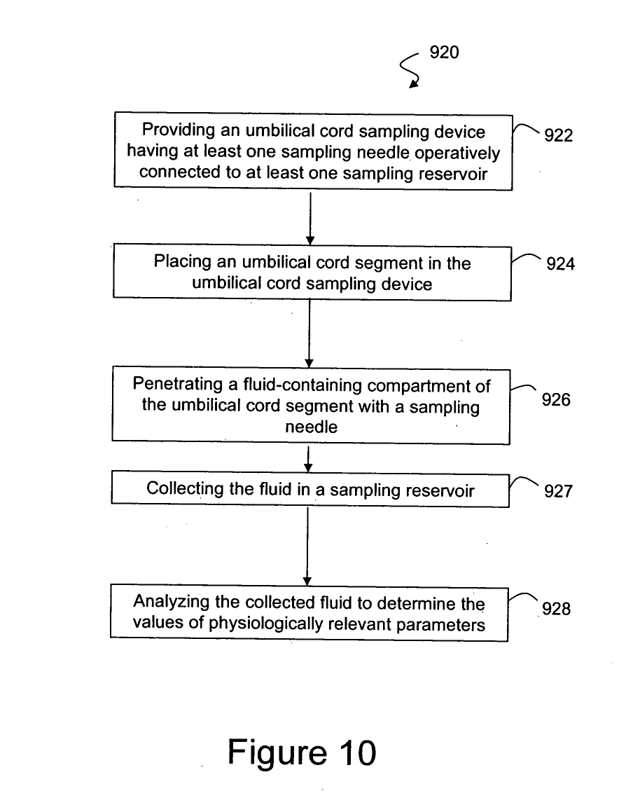

FIG. 10 is a schematic diagram illustrating a method 920 for determining the values of physiologically relevant parameters of a biological fluid, comprising the steps of providing an umbilical cord sampling device having at least one sampling needle operatively connected to at least one sampling reservoir 922; placing an umbilical cord segment in the umbilical cord sampling device 924; penetrating a fluid-containing compartment of the umbilical cord segment with a sampling needle 926; collecting the fluid in a sampling reservoir 927; and analyzing the collected fluid to determine the values of physiologically relevant parameters 928. In some embodiments, the analyzing step of the method further comprises the step of transferring a portion of the sample to a clinical laboratory for further analysis. In some embodiments, the analyzing step of the method further comprises the step of communicating analysis results to a computer system. In some embodiments, the method further comprises the step of storing an aliquot of cord blood.

In embodiments in which the collected fluid is blood, analysis of the collected blood is performed using one or more of the following tests: ABO blood type and antiglobulin (Coomb's Antibody) to determine baby's blood type and whether or not the maternal immune system has passed any antibodies to the baby, blood gases and pH, respiratory status, electrolytes, complete blood count (CBC), platelet count, hemoglobin levels (Hgb), hematocrit (HCT), bilirubin levels, glucose, lead, TSH, PKU, toxicology and blood culture (if an infection is suspected), depending on the circumstances. In some embodiments, a sample of blood, or a sample DNA extracted from an aliquot of blood, can be stored for later use, e.g., identification of the patient.

The claims should not be read as limited to the described order or elements unless stated to that effect. Therefore, all embodiments that come within the scope and spirit of the following claims and equivalents thereto are claimed as the invention.

1. An umbilical cord fluid sampling device comprising:
at least one moveable sampling needle;
a needle housing constructed and arranged to support the at least one moveable sampling needle;
a base housing constructed and arranged to support a portion of the umbilical cord of a patient, at least one of the base housing and the needle housing being moveable between a closed position and an open position in order to insert the umbilical cord therein;
an interior chamber disposed between the needle housing and the base housing;
at least one sampling reservoir supported within a portion of the chamber and in fluid communication with the at least one moveable sampling needle; and
an opening disposed through at least one of the base and needle housing and in fluid communication with the sampling reservoir, the opening being configured and dimensioned to receive the removable cassette such that a fluid sample may be removed from the sampling reservoir while the base and needle housing remain in the closed position.

2. The umbilical cord sampling device of claim 1 wherein the opening is a slot in fluid communication with the sampling reservoir, the slot being dimensioned to receive a portion of a removable cassette.

3. The umbilical cord sampling device of claim 2, wherein the cassette includes a deposition well and the slot is disposed within the sampling device such that the deposition well is positioned in fluid communication with the reservoir upon insertion of the cassette so that a sample of the fluid is transported from the at least one sampling reservoir to the deposition well upon insertion of the cassette.

4. The umbilical cord sampling device of claim 3, wherein the reservoir further includes a valve constructed and arranged to restrict flow of the fluid from the reservoir, the valve being aligned with the slot such that the valve is activated when the cassette is inserted and engages the valve.

5. The umbilical cord sampling device of claim 4, wherein the valve is a ball valve disposed within a conduit that provides fluid communication between the reservoir and slot, the ball valve being biased into a closed position by a spring so as to restrict the flow of fluid through the conduit in the closed position.

6. The umbilical cord sampling device of claim 1 wherein the opening is a port that is configured and dimensioned to receive a syringe therein.

7. The umbilical cord sampling device of claim 1 further comprising a roller.

8. The umbilical cord sampling device of claim 1 wherein at least one sampling needle is positionable relative to an umbilical cord segment within the device.

9. The umbilical cord sampling device of claim 8 further comprising at least one knob operatively connected to the at least one sampling needle so as to move the needle in a transverse direction with respect to a longitudinal axis of the cord.

10. The umbilical cord sampling device of claim 8 further comprising a plunger constructed and arranged to move the at least one sampling needle into the umbilical cord.

11. The umbilical cord sampling device of claim 1 in combination with an analyzer unit constructed and arranged to receive the removable cassette so as to perform fluid analysis on the sample removed from the at least one reservoir.

12. The device of claim 1 wherein the at least one sampling needle comprises two sampling needles and a pair of knobs for moving the sampling needles in a transverse direction with respect to a longitudinal axis of the cord and a plunger for inserting the needles into the cord.

13. The device of claim 1 further comprising a window positioned so as to view the sampling needles during use.

14. A method for determining the values of physiologically relevant parameters of a biological fluid, comprising the steps of:
providing an umbilical cord sampling device having at least one moveable sampling needle operatively connected to at least one sampling reservoir;
placing an umbilical cord segment in the umbilical cord sampling device;
penetrating a fluid-containing lumen of the umbilical cord segment with a moveable sampling needle;
collecting the fluid in a sampling reservoir; and
removing the collected fluid from the sampling reservoir for analysis.

15. The method of claim 14 further comprising the step of providing a valve to restrict fluid flow from the reservoir and an opening in the sampling device in fluid communication with the reservoir, the opening being configured and dimensioned to receive one of a syringe or cassette.

16. The method of claim 14 further comprising the step of inserting the cassette into the opening and into engagement with the valve so as to allow fluid to flow from the reservoir to the cassette.

17. The method of claim 16 further comprising the step of removing the cassette from the opening and inserting it into an analyzer.

18. The method of claim 13 further comprising measuring a blood analyte.

19. An umbilical cord fluid sampling device comprising:
at least one moveable sampling needle;
a needle housing constructed and arranged to support the at least one moveable sampling needle;
a base housing constructed and arranged to support a portion of the umbilical cord of a patient, at least one of the base housing and the needle housing being moveable between a closed position and an open position in order to insert the umbilical cord therein;.
an interior chamber disposed between the needle housing and the base housing;
at least one sampling reservoir supported within a portion of the chamber and in fluid communication with the at least one moveable sampling needle;
a removable cassette including a base, a cover, a deposition well constructed and arranged to receive fluid therein and a calibrant solution supported within the cassette;
an opening disposed through at least one of the base and needle housing and in fluid communication with the sampling reservoir, the opening being configured and dimensioned to receive the removable cassette such that a fluid sample may be removed from the sampling reservoir while the base and needle housing remain in the closed position.

20. The umbilical cord sampling device of claim 19, wherein the calibrant solution is disposed within a pouch and the cassette further includes a barb for releasing the solution from within the pouch upon insertion within an analyzer.
